(19) United States
(12) Patent Application Publication
(10) Pub. No.: US 2016/0270694 A1
(43) Pub. Date: Sep. 22, 2016

(54) Laparoscopic Device and Method

(71) Applicant: Medlinx Acacia Pte. Ltd., Singapore (SG)

(72) Inventors: Joseph Tang, Shanghai (CN); L. Thomas Divilio, Easton, MD (US); Yin Chiang Freddy Boey, Singapore (SG)

(21) Appl. No.: 15/076,215

(22) Filed: Mar. 21, 2016

Related U.S. Application Data
(60) Provisional application No. 62/135,217, filed on Mar. 19, 2015.

(51) Int. Cl. A61B 5/107; A61B 1/005; A61B 1/313; A61B 17/00; A61B 1/00

(52) U.S. Cl. CPC A61B 5/1076; A61B 17/00234; A61B 1/00087; A61B 1/3132; A61B 1/00052; A61B 1/0052

(57) ABSTRACT

A laparoscopic tool, the tool including an elongate shaft; a handle at one end of the shaft and an aperture at the opposed end, and; a tape selectively extendable from the aperture.

### CROSS-REFERENCE TO RELATED APPLICATIONS

This application claims priority to and the benefit of U.S. Provisional Patent Application Ser. No. 62/135,217, filed Mar. 19, 2015, entitled “Improved Laparoscopic Device and Method,” by Joseph Tang, et al. which is incorporated herein by reference in its entirety for all purposes.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND

The invention relates to apparatus and methods used in the identification and analysis of tears, fissures and other defects in tissue, animal, human or otherwise. In particular, the invention relates to the measurement of said defects for subsequent treatment.

Whilst methods exist to measure tissue defects, these are cumbersome and time consuming. Accuracy is also an issue, particularly when direct measurement is required. A defect within the abdomen when viewed through a laparoscopic endoscope distorts the visualization of this defect.

There is currently no single device that can be used to take a linear measurement within the body laparoscopically. Linear measurements are currently taken by inserting a surgical measuring tape within the body and using two graspers to extend the tape. The measurement is then read from the tape through an endoscope, which could distort the reading of the measurement. This current method is cumbersome and time-consuming.

### SUMMARY

In a first aspect, the invention provides a laparoscopic tool, the tool including an elongate shaft; a handle at one end of the shaft and an aperture at the opposed end, and; a tape selectively extendable from the aperture.

Accordingly, the invention provides a laparoscopic endoscope having an integral measurement device, operable from a handle of said laparoscopic endoscope. By incorporating the measurement device, the operator/surgeon is able to take measurements more efficiently and consequently more accurately.

The laparoscopic endoscope may also include means to read the measurement from the handle, as compared to viewing the measurement through an endoscope, and so avoiding possible distortion.

In this embodiment, the measurement may be in the form of an analogue or digital display.

The measurement device may include a filament projecting from the laparoscopic endoscope in order to make the measurement. To this end, the filament may be a stainless steel tape, which may have gradations to permit reading the measurement through an endoscope.

Alternatively, the filament may be a wire, with the distance the wire projects from an end of the laparoscopic endoscope measured and displayed on the handle. Alternatively, the filament may be a linear array of balls connected through a wire so as to allow articulation between adjacent balls. The connecting wire may be pre-tensioned so as to apply a pre-load to the balls, and so aiding in the stiffness of the linear array. This may consequently assist in positioning the linear array in the desired position for measurement.

The laparoscopic tool may be intuitively easy to use (single operator and single-handed operation best), but not precluding the use of two-handed operation.

Fits directly into current operating workflow

Cost effective (an affordable single use tool)

Accurate to +/−1 mm

Safe for use in the operating environment

### BRIEF DESCRIPTION OF THE DRAWINGS

It will be convenient to further describe the present invention with respect to the accompanying drawings that illustrate possible arrangements of the invention. Other arrangements of the invention are possible, and consequently the particularity of the accompanying drawings is not to be understood as superseding the generality of the preceding description of the invention.

FIG. 1 is a schematic view of a laparoscopic endoscope according to one embodiment of the present invention;

FIG. 2 are various views of a laparoscopic endoscope according to a further embodiment of the present invention;

FIG. 3 is a sectional view of the laparoscopic endoscope of FIG. 2;

FIGS. 4 and 5 are isometric views of the laparoscopic endoscope of FIG. 2;

FIG. 6 are sequential views of a laparoscopic endoscope in use according to a further embodiment of the present invention;

FIG. 7 is a sectional view of a laparoscopic endoscope according to a further embodiment of the present invention;

FIG. 8 is various views of laparoscopic endoscopes according to several embodiments of the present invention;

FIG. 9 is an isometric view of a laparoscopic endoscope according to a further embodiment of the present invention;

FIG. 10 is a flow chart detailing interactions of a laparoscopic endoscope according to a further embodiment of the present invention;

FIG. 11 is a sectional view of a laparoscopic endoscope according to a further embodiment of the present invention;

FIG. 12 are various views of a laparoscopic endoscope according to a further embodiment of the present invention;

FIG. 13 is an isometric view of a laparoscopic endoscope according to a further embodiment of the present invention;

FIG. 14 is a schematic view of the operation of a laparoscopic endoscope according to a further embodiment of the present invention;

FIG. 15 is a schematic view of the operation of a laparoscopic endoscope according to a further embodiment of the present invention;

FIG. 16 are various views of a laparoscopic endoscope according to a further embodiment of the present invention;

FIG. 17 are various views of a laparoscopic endoscope according to a further embodiment of the present invention;

FIG. 18 are various views of an alternate application of the laparoscopic endoscope FIG. 17;

FIGS. 19 to 28 are various embodiments of laparoscopic positional devices.

### DETAILED DESCRIPTION

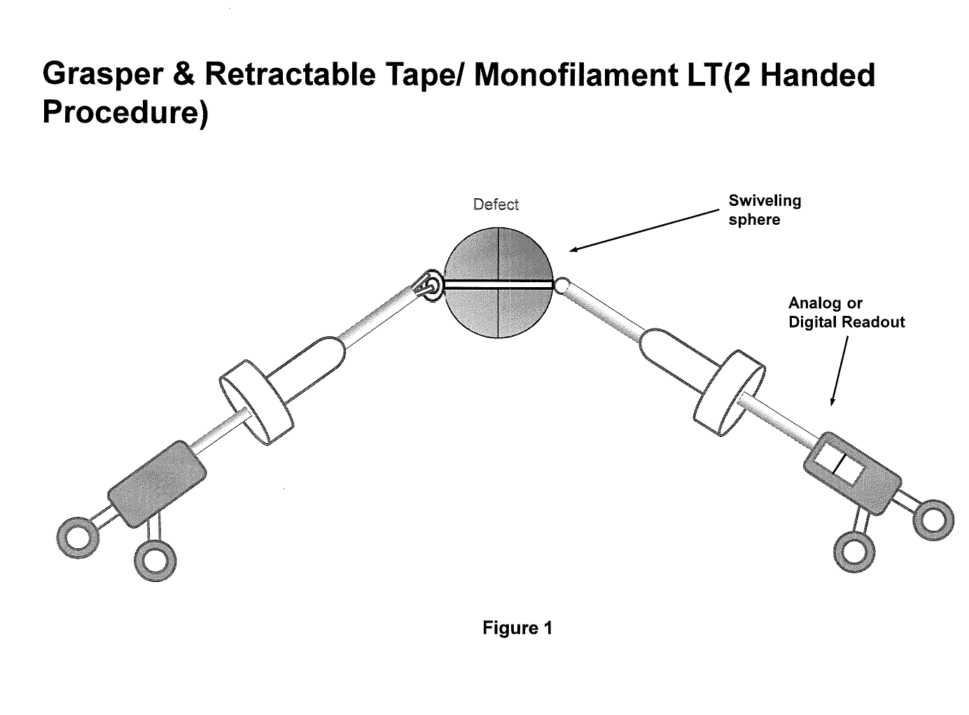

As shown in FIG. 1, a procedure 5 using a laparoscopic endoscope 10 and a grasper 15 are used by the surgeon to measure the size 42 of a defect 20 (such as a hernia). In this embodiment, the laparoscopic endoscope 10 includes a retractable measuring tape/steel monofilament 40, with a ring 35 attached to the end of the tape 40 and places it across the defect 20. The width 42 of defect 20 can either be read from the tape itself or via an analog or digital readout on the laparoscopic endoscope 10. In this embodiment, the end of the laparoscopic endoscope 10 includes a swiveling tip to direct the tape 40 in the desired direction.

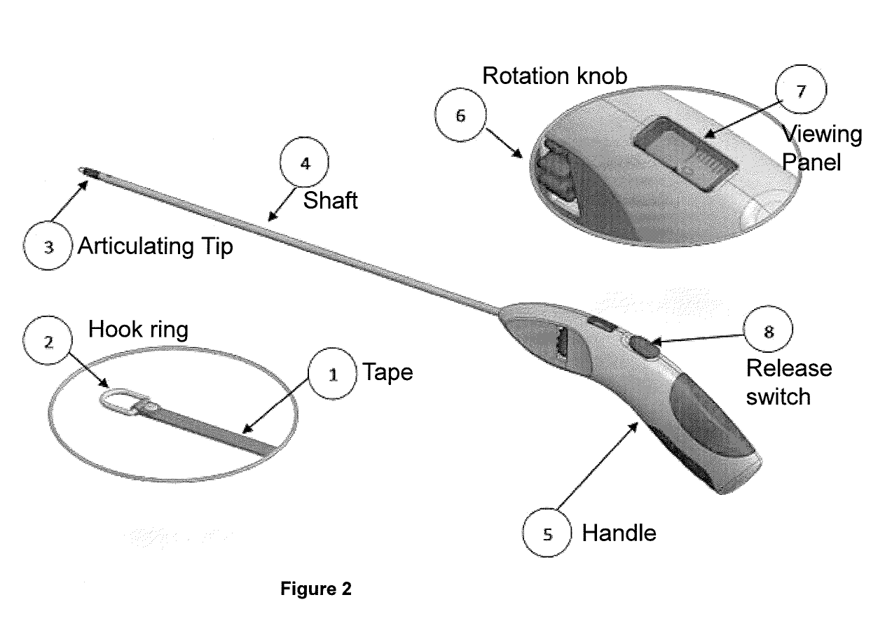

As shown in FIG. 2, one embodiment of the present invention includes is a single use laparoscopic surgical tool, used to provide size information of a hernia defect to a surgeon in order to assist in determining the size of a hernia polymeric mesh. It is also useful in determining the size of an intra-abdominal mass, or other lesion, when laparoscopic techniques are used.

The laparoscopic endoscope includes a measurement tape 45 with a hook ring 50, an articulating tip 55, a shaft 60 and handle 65. In this particular embodiment, the measurement tape 45 can be extended to a maximum length of 20 cm, but it will be appreciated that the length of the tap 45 is provided by way of example and is not limiting on the scope of the invention.

The tape 45 is enclosed by the handle 65 and the shaft 60, and can be extended out by grasping the hook ring 50 with a laparoscopic grasper tool (not shown), and pulling it away from the handle or instrument tip. The shaft can be rotated up to 330 degrees using the rotation knob 70. The extended tape would be aligned to the hernia defect for length (diameter) information through the Viewing Panel 75. The tape can be retracted by pushing the switch 80 forward.

The device works on spring loaded spool and ratchet mechanism for extending and retracting the tape. The ratchet will be affective when extending the tape, while a rotary damper will slow down the retraction of the tape by the spool.

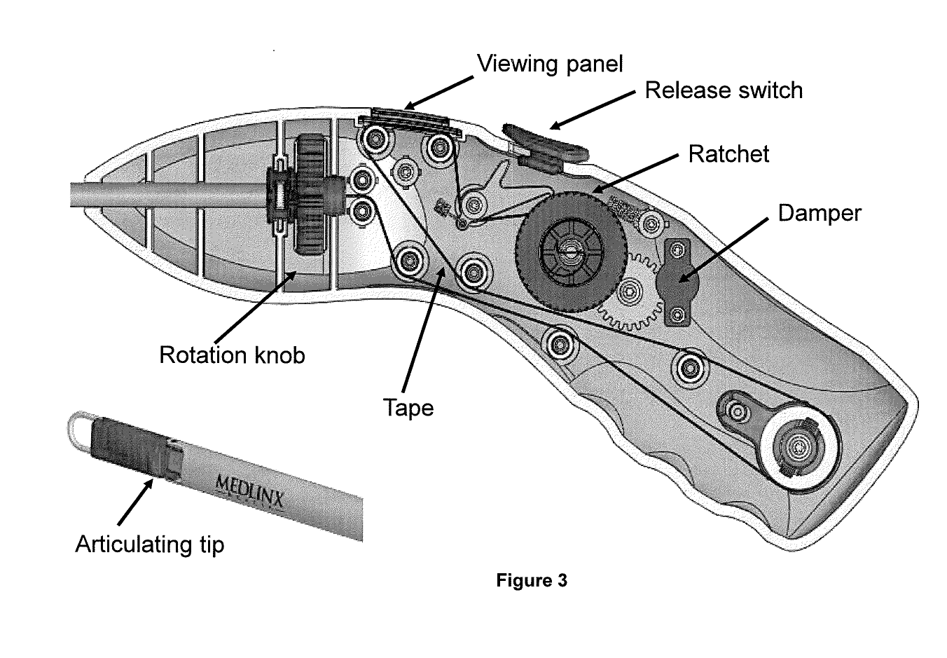

FIG. 3 shows the internal arrangement for one laparoscopic device 85 according to the present invention. As before the device 85 includes a viewing panel 90 for displaying the extent of the filament (or tape 110, in this case) projecting from the tip 120. In this case, a portion of the filament extends into the handle, with the base line on the filament indicating against a data, visible through the viewing panel 90, such that as the filament extends, the length can be read as an analogue measurement. There is a release switch 95 for selectively locking the filament and a ratchet from preventing an uncontrolled retraction of the filament, which is biased to retract on release of the switch 95.

There is also a damper 105 to protect the device from over extension or retraction of the tape 110.

Further, the shaft through which the tape passes is rotatable using a rotation knob 115. This is particularly useful for aligning the tape against the defect, and works with the articulating tip to provide pivotal movement of the tip about the longitudinal axis of the shaft.

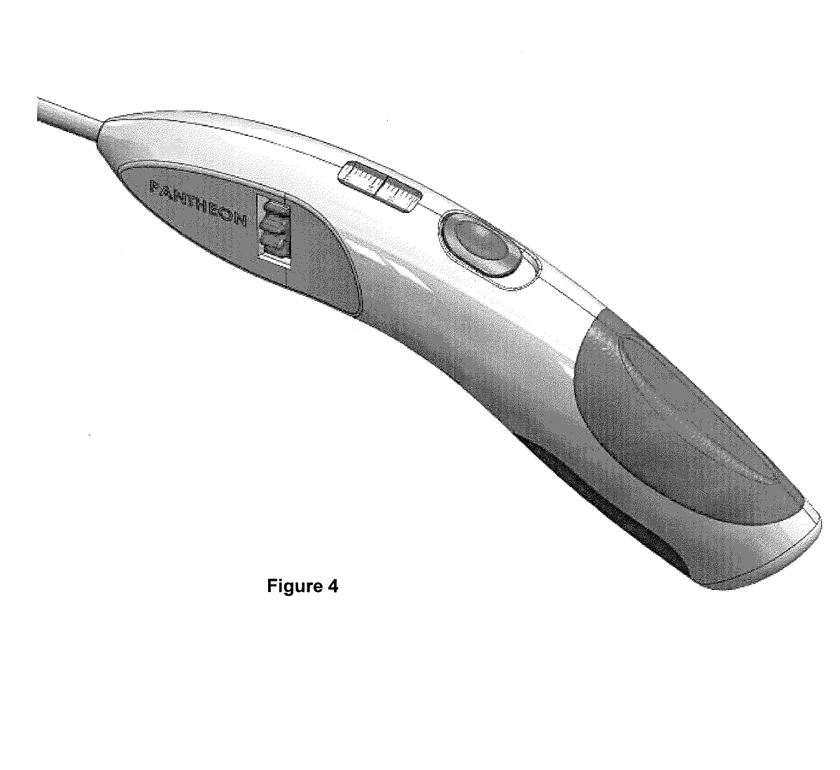

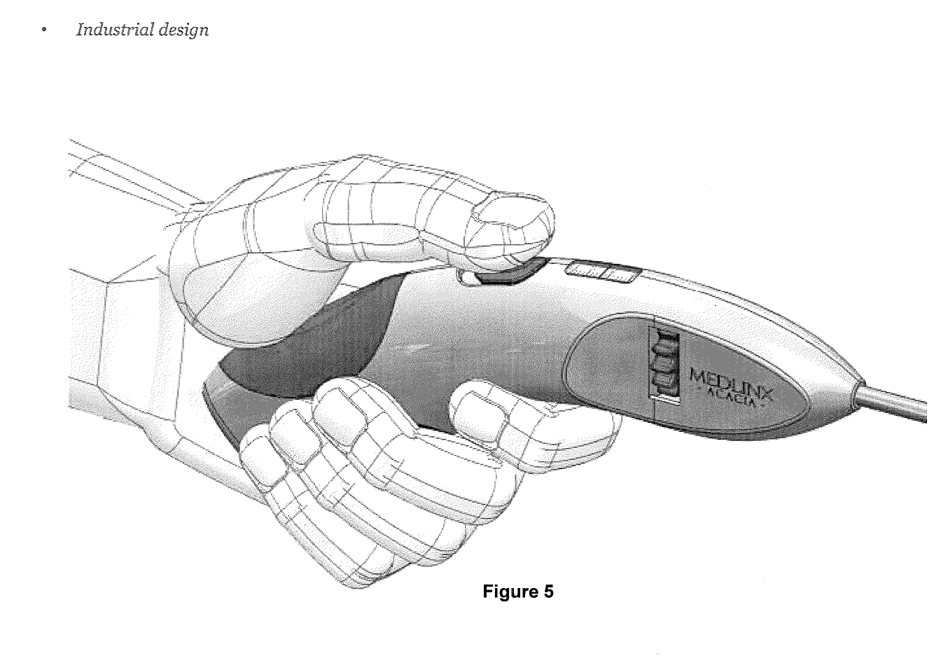

FIGS. 4 and 5 show the arrangement and handling of the device 85 of FIG. 3. Importantly, the arrangement of the device, whereby one handed operation 125 is permitted can be seen in FIG. 5.

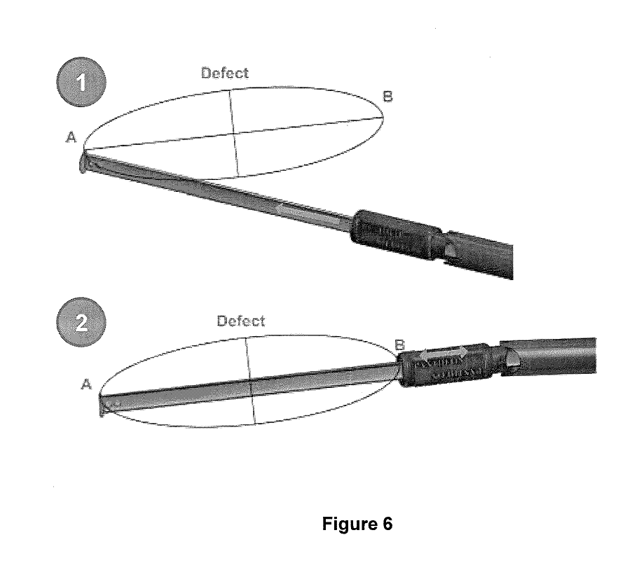

Referring to the measurement workflow 130 of FIG. 6, the use of the device includes the steps of over-extending 150 the filament 145 and align edge of tape measure to point A, then while edge of tape is anchored at point A, move tip of device towards point B and extend/retract 155 filament to align with point B.

The position of the tip is facilitated in this embodiment by the use of an articulation of the tip 140 relative to the shaft 135.

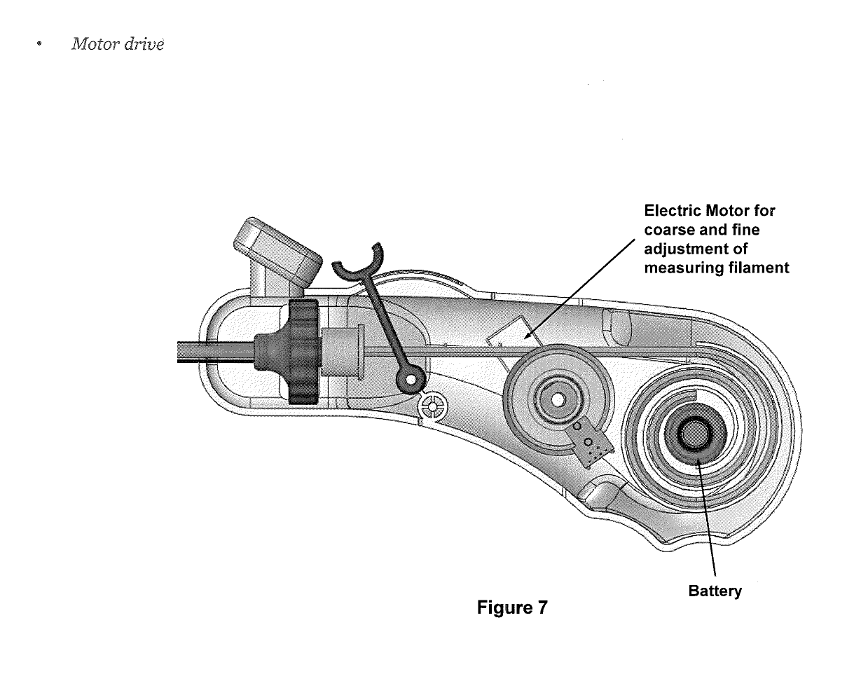

FIG. 7 shows other possible arrangements to the device 170, including a lever 175 which is connected to two guide wires running the length of the shaft and connecting to the articulated tip (not shown). By moving the lever 175, the guide wires allow pivoting of the tip within a plane, defined by the placement of the guide wires on opposed sides of the tape/filament.

To facilitate control of the filament retraction/extension, the device 170 of FIG. 7 includes a motor 180, powered by a battery 185.

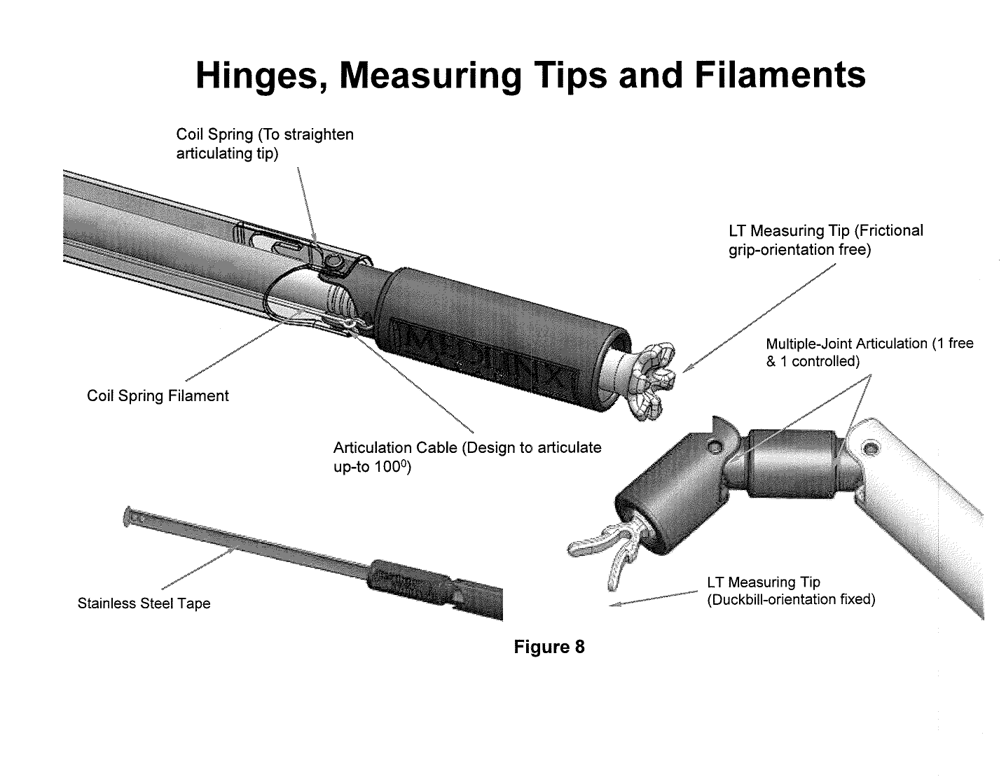

FIG. 8 shows various embodiments, particularly regarding the tip.

One embodiment of the tip 155 shows guide wires 215 as previously described, placed on opposed sides of the filament and connected to the tip 195. By applying a tensile force to one guide wire, the articulated tip 195 is biased, within the laner defined by the guide wires, in the respective direction. On release of the force, a spring 205 biases the tip 195 back to the central position. In one embodiment, the articulated tip may be pivoted up to 1 pp degrees from the longitudinal axis of the shaft.

Attached to the end of the tape, but not limited for use with this particular embodiment, is a frictional grip 200. Having a plurality of minor projections, the grip 200 is arranged to engage the tissue so as to anchor the tape. In this way, a grasper may not be required allowing the surgeon to conduct the measurement one handed.

An alternative grip 235 is shown, having a Y shape, and arrange to engage the tissue in a frictional engagement or alternatively to clamp or pinch flaps of tissue in order to anchor the tape.

This diagram also shows an embodiment whereby the tip has multiple articulations, in particular a linkage 225, 227 joined by a hinge and projecting from the shaft 223. With the guide wires connected to the end link 227, the intermediate link 225 does not need to be controlled and the hinges 224, 230 providing a far greater degree of pivoting angle.

It will be appreciated that the grips and tip arrangements are now limited for use with the corresponding features shown in FIG. 8. Each of the features may be used separately, with FIG. 8 providing an illustrative view of each.

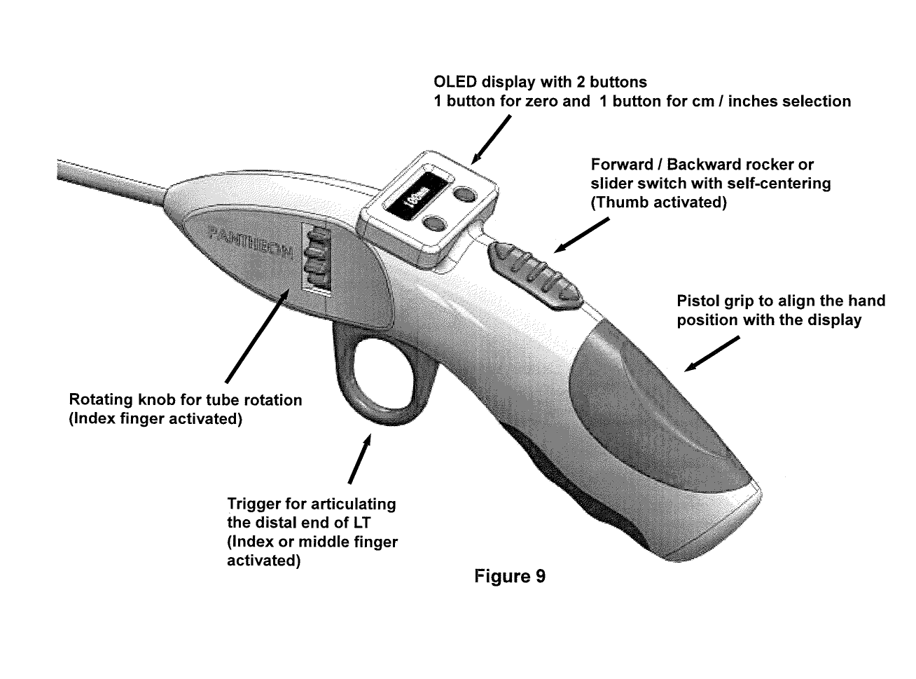

FIG. 9 shows a further embodiment of a handle 240 according to the present invention.

A digital display 245, with a zero button is provided, together with a toggle between inches and millimetres. The handle includes a pistol grip 255, with a trigger 260 for the articulation of the tip, the trigger connected to guide wires for reciprocal movement arranged to reciprocally pivot the tip.

A rotating knob 265 is also provide, and directly connected to the shaft for rotating the shaft. Rotation of the shaft together with pivoting of the tip allows for full articulation about the longitudinal axis of the shaft. A “rocker” switch 250 is also provided for incrementally moving the tape backward and forward as a means of fine control. The switch is biased to a central position to facilitate said movement.

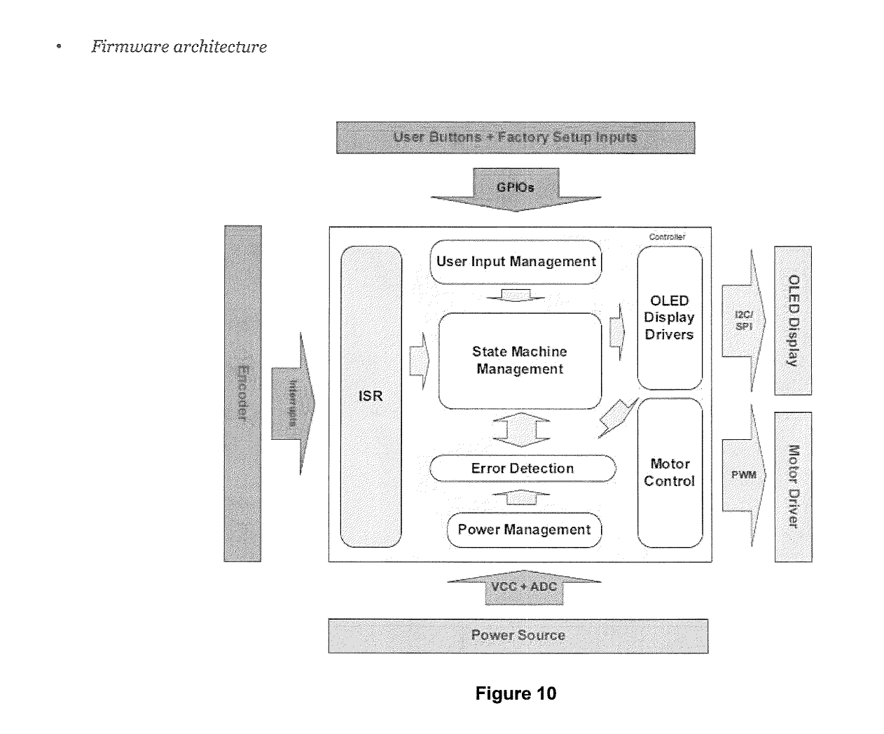

FIG. 10 shows a flow chart of input and output from the device.

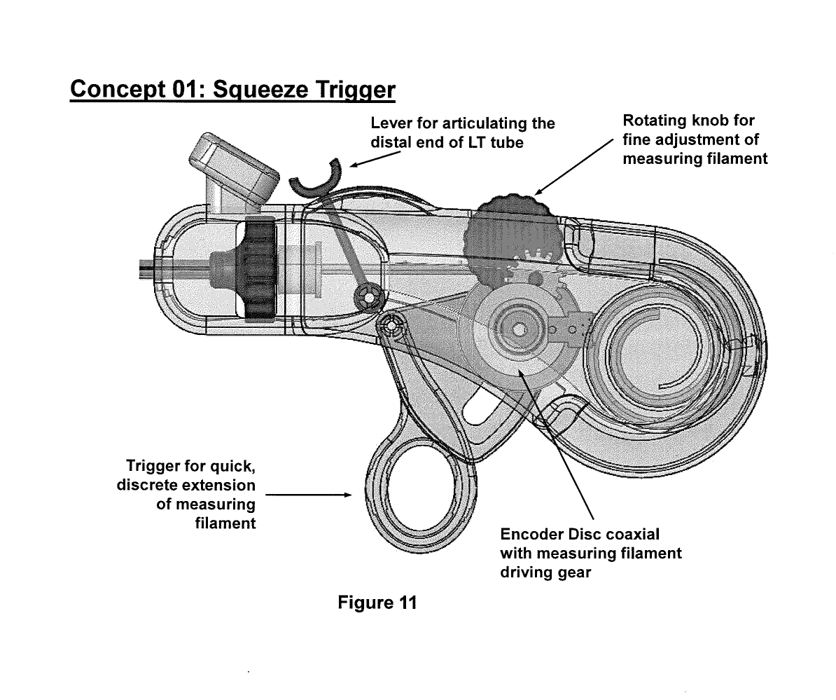

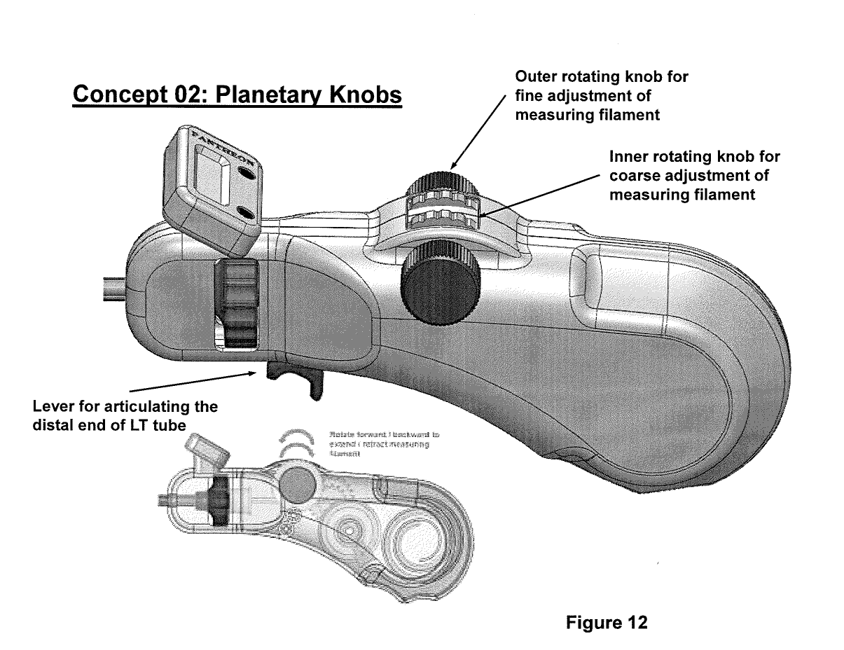

FIG. 11 shows a further embodiment of the present invention. In particular is shows a handle 270 having a lever 275 connected to guide wires for articulating the tip. Rapid deployment of the tape is provided by a trigger 290, with fine adjustment provided by a rotating knob 280. Measurement of the extended tape is achieved by an encoder 285 tacking the movement of the tape within the handle. This compares to a rearrangement 295 of features as shown in FIG. 12 with the coarse and fine adjustment provided by adjacent knobs 300, 305, and the lever provided on an underside of the handle 295.

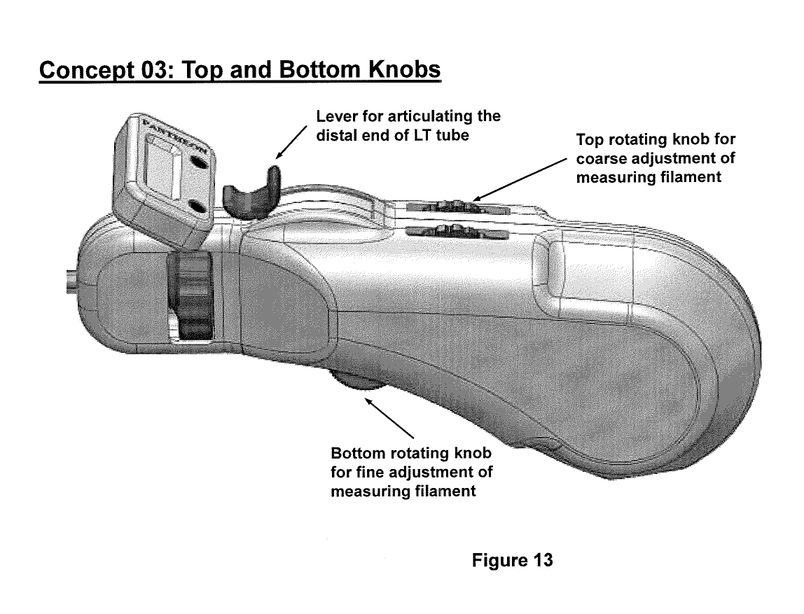

FIG. 13 shows a still further rearrangement 315 with an amended form of the fine 330 and coarse adjustment knobs 325 on respective bottom and top portions of the handle 315. A lever 320 similar to that previously described is also shown adjacent to the display panel on the top portion.

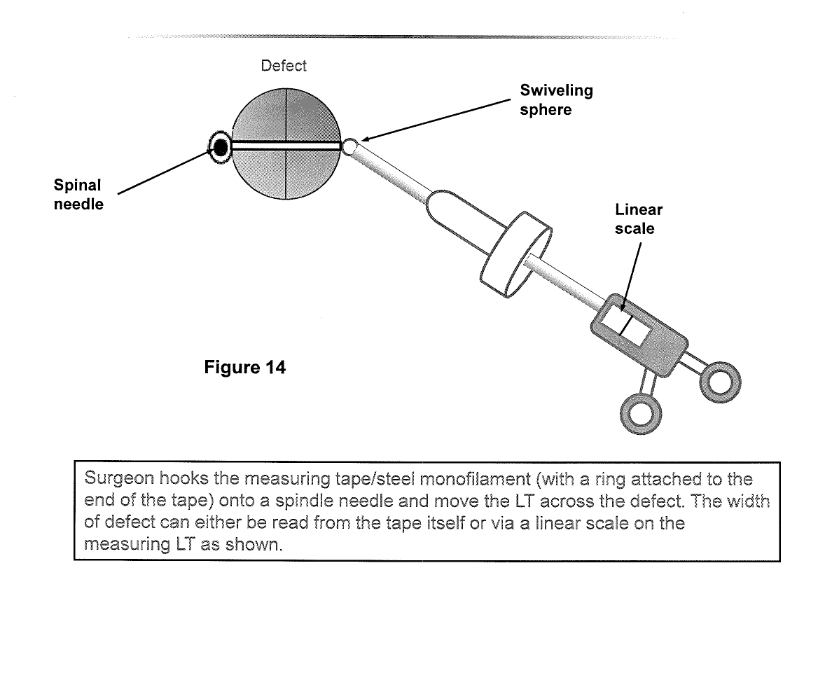

FIG. 14 shows one method of measurement of a defect in tissue, such as a hernia 350, whereby a device 340 according to one embodiment of the present invention has a display on the respective handle. Here a spinal needle 335 is placed in the tissue at a point adjacent to one side of the hernia. The tape engages the needle through a hook or loop 360 and the tape is drawn out of the shaft until it aligns, through a swiveling tip 345, with the opposed edge of the hernia. The length 362 can then be read from the display 335, or directly from gradations on the tape.

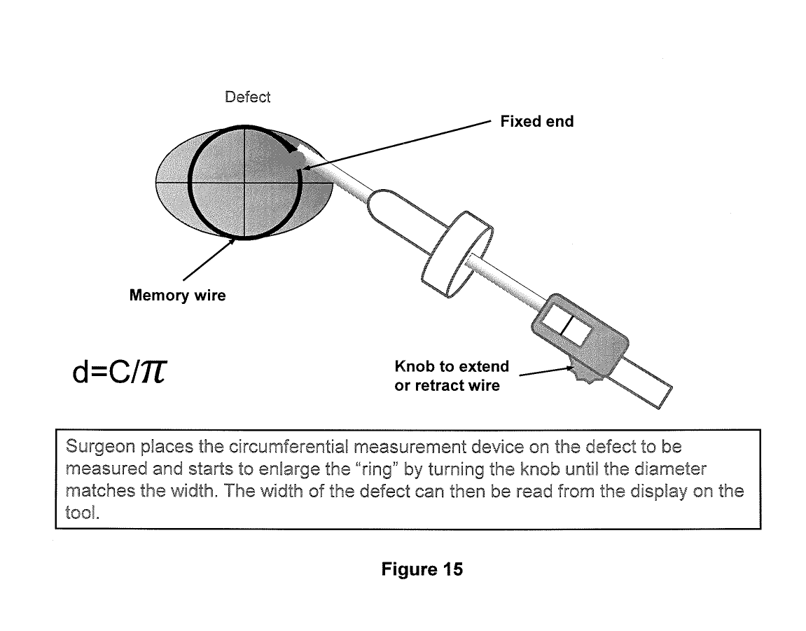

A slightly more indirect method is shown in FIG. 15, whereby anchoring the needle 380 in the tissue does not correspond to a convenient point of measurement. By placing the needle where convenient the tape, in this case a memory wire, can be formed into a loop, with a diameter of the loop corresponding to a dimension 395 of the hernia 390. The displayed length will provide the circumference with the diameter 395 readily calculated.

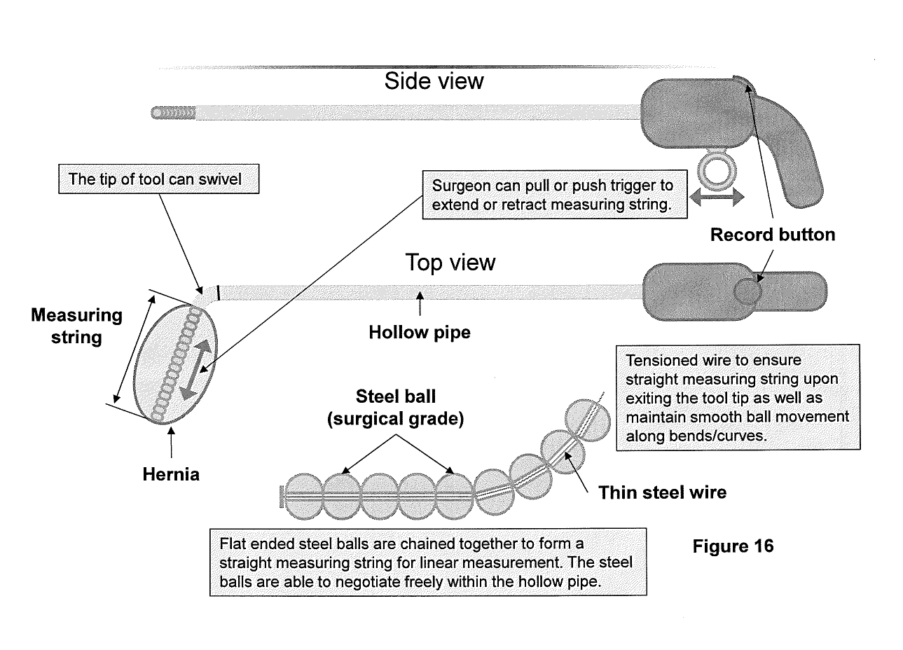

FIG. 16 shows a further embodiment of the present invention. A laparoscopic device 400 having a handle corresponding to any of the previous embodiments, includes a filament comprising a linear array 420 of steel balls 430 connected by a wire 435 passing through the centre. The balls may include flat portions 450 to provide stable abutting surfaces between adjacent balls. Further, the wire may be pre-tensioned to apply a pre-load to the balls, increasing the stiffness of the linear array.

Extending 445 the linear array 420 may be according to any previous embodiment, and in this case includes a trigger 405, and may include a recording button 415 for recording the measurement, and an articulated tip 440. The articulation of the adjacent balls facilitates the position of the linear array so as to align the filament across of convenient dimension 425 of the hernia.

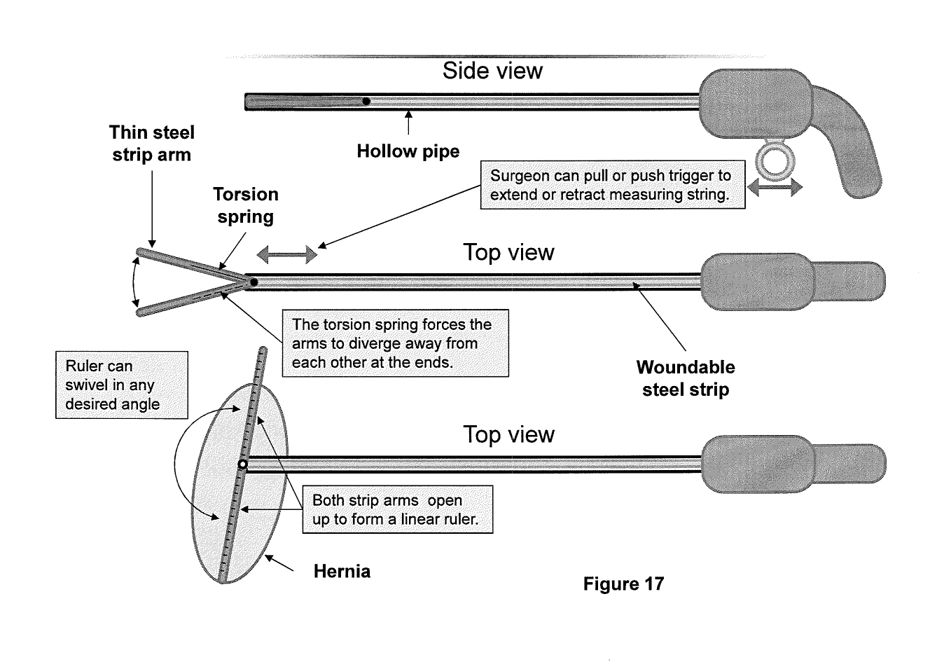

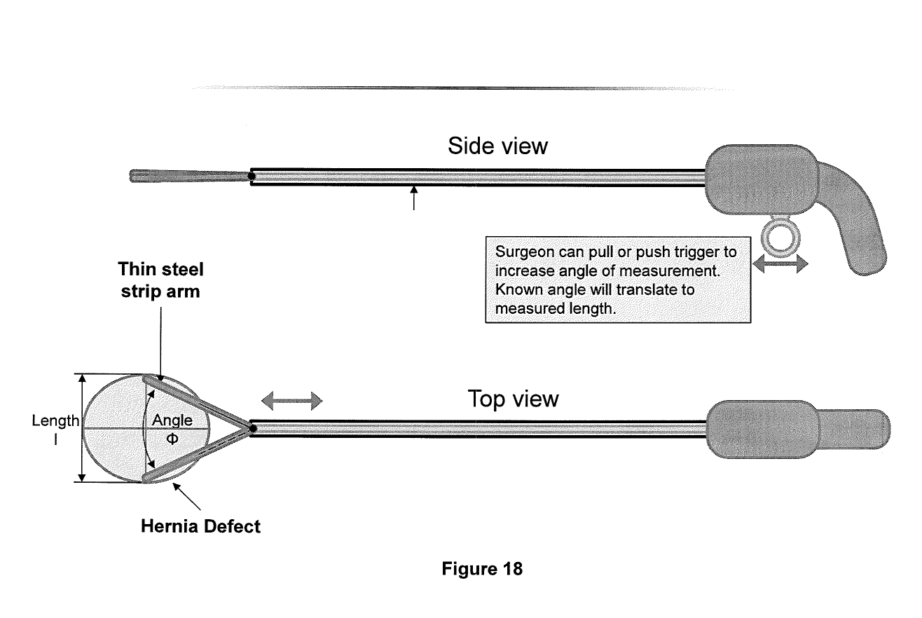

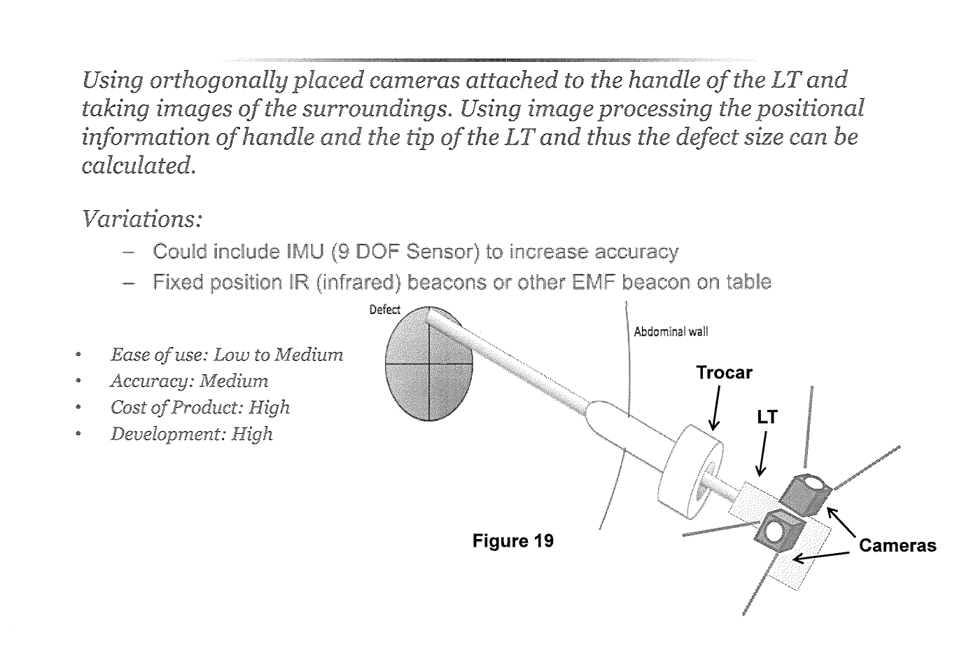

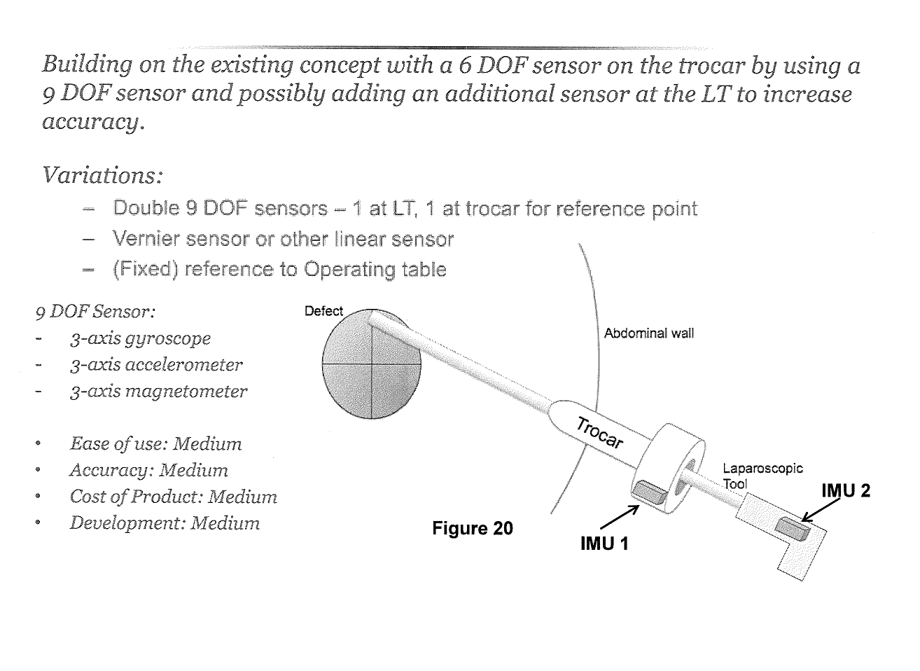

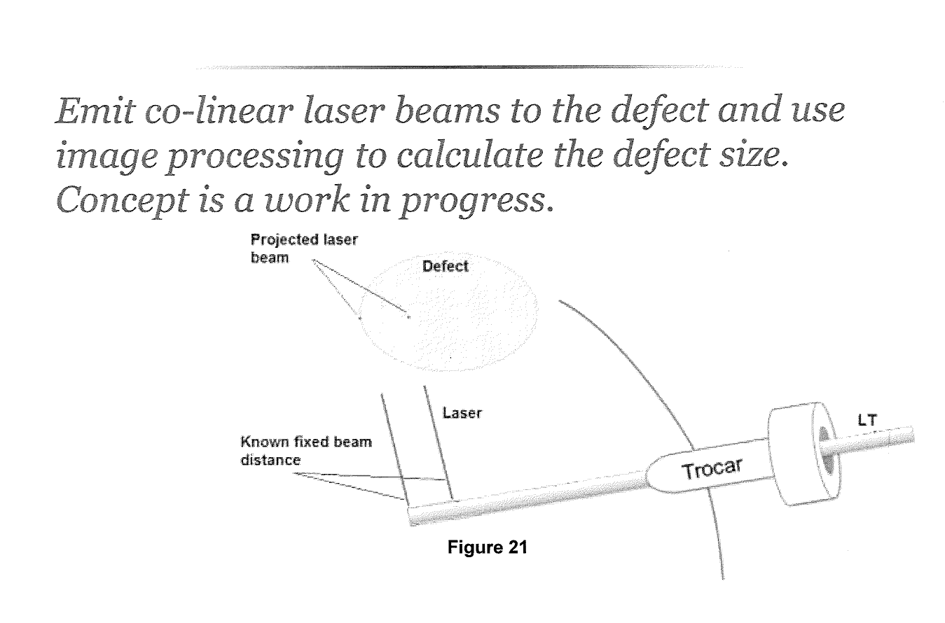

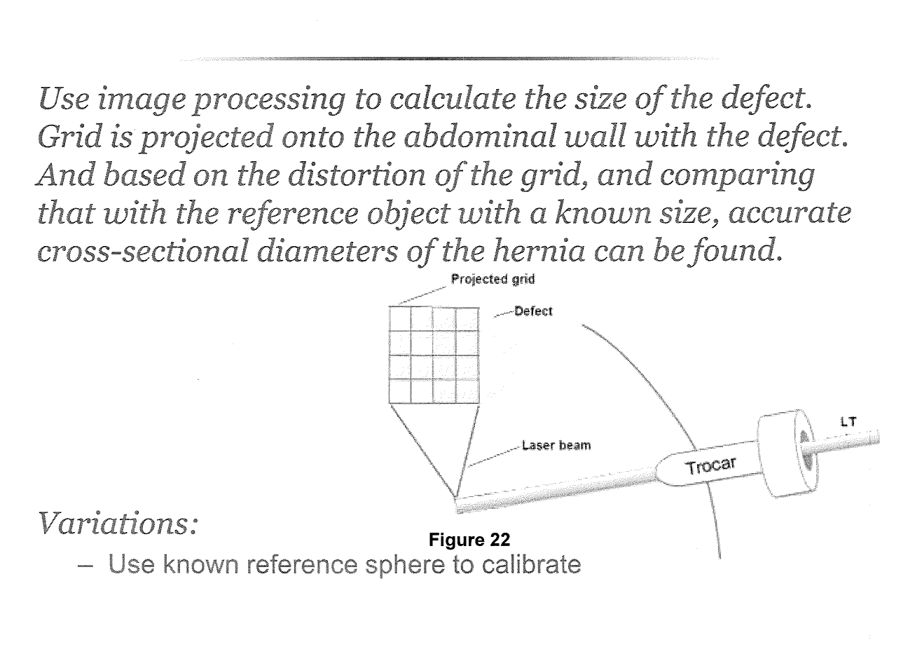

FIGS. 17 and 18 show a still further embodiment, with a device 455 having a handle 460 and shaft 465 of the present invention. Here the measuring portion of the filament is a rigid linkage 470, comprising a 1^{st} and 2^{nd} 472, 475 portion having an intermediate hinge 495 in the linkage. In this case, the hinge is spring loaded. Extending 469 the linkage from the shaft 465, permits the linkage to spring into a linear ruler for direct measurement of the hernia 485. The length of the fissure can then be readily measured by observation. On extension, the hinge 495 is adjacent to the tip 490, and so applying a retraction force pivots the linkage so as to fit back within the shaft 465.

FIG. 18 shows a similar arrangement, however extension 505 of the linkage 500 is only allowed so as to form a V shape, having an angle 515 between the 1^{st} and 2nd links. Respective ends of the 1^{st} and 2^{nd} links 520, 525 are then positioned at convenient edges of the hernia 535 and the angle measured by observation. As the 1^{st} and 2^{nd} links are of the same length, the length 530 of the hernia can then be readily calculated.

CMM is the Coordinate-measuring machine that is used to measure accurately points in space and distances. Its accuracy is due to a stable base and hinges/linkages that are able to tell the angle moved.

Variations:

Mechanical

Optical/wireless linkages

Use spheres of known size as references and capture and compare both the hernia defect and spheres in the photo.

Variations:

Image process the size to give positional info

Ease of use: Medium to Low

Accuracy: Medium

Cost of Product: Low

Development: Medium (mainly image recognition and app development)

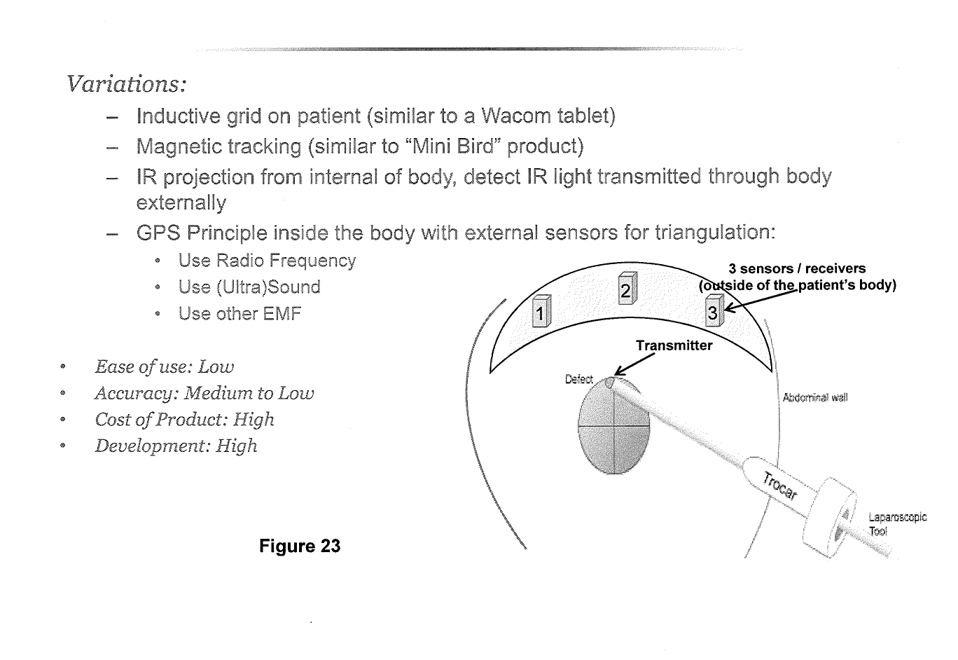

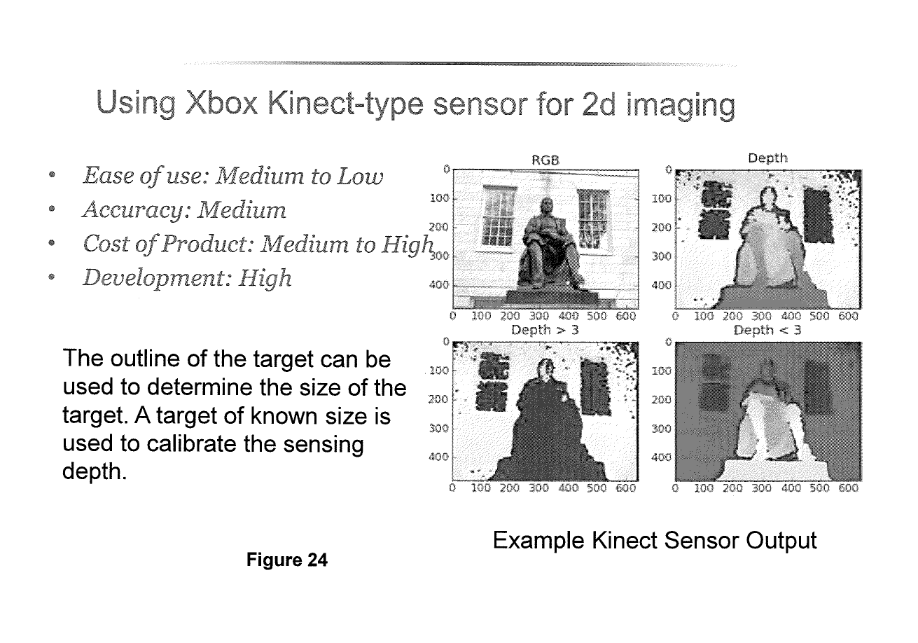

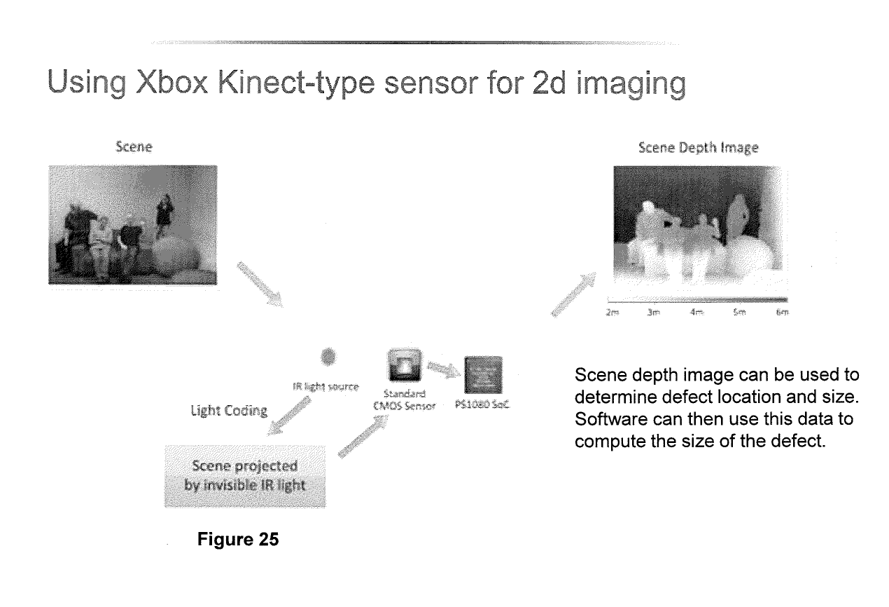

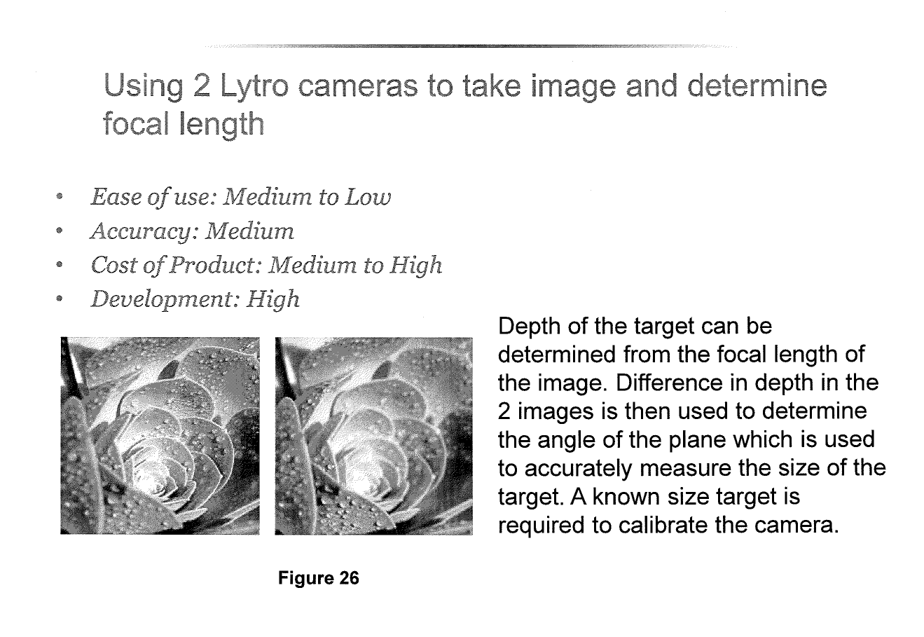

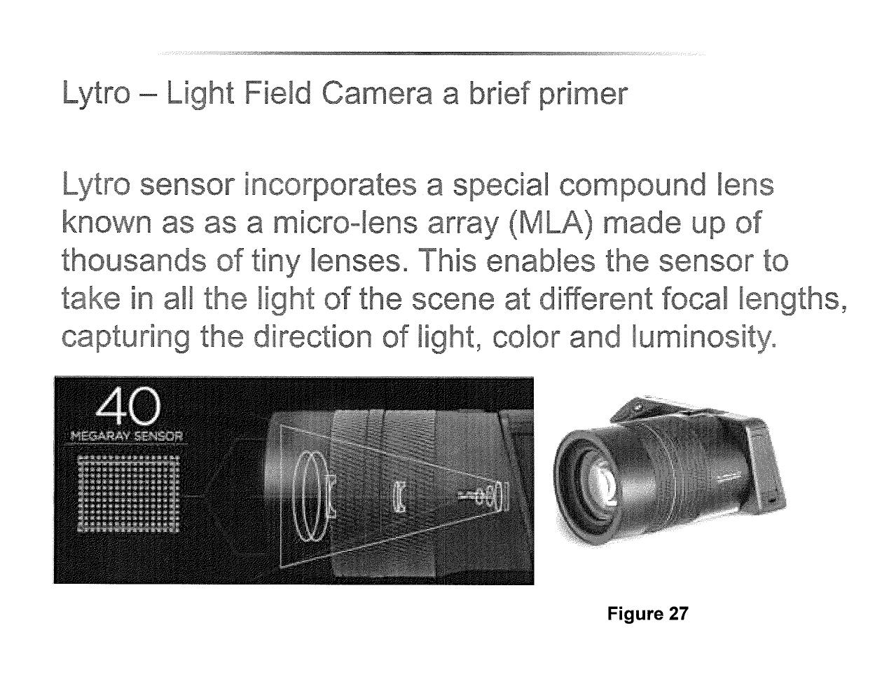

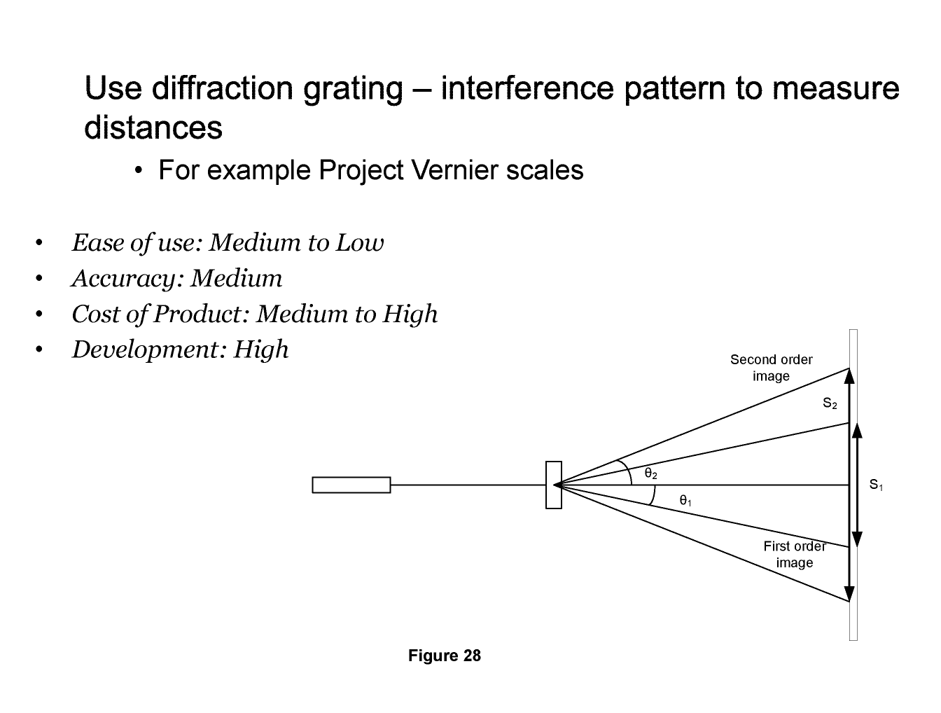

Variations for FIG. 23:

Xbox Kinect-type sensor for 2d imaging

Use 2 Lytro cameras to take image and determine focal length

Use diffraction grating—interference pattern to measure distances

For example Project Vernier scales

Ease of use: Medium to Low

Accuracy: Medium

Cost of Product: Medium to High

Development: High

What is claimed is:

1. A laparoscopic tool, the tool including
an elongate shaft;
a handle at one end of the shaft and an aperture at the opposed end, and;
a tape selectively extendable from the aperture.

2. The tool according to claim 1, further including a measuring device arranged to measure a length of the extended tape.

3. The tool of claim 1, wherein the measuring device is in communication with a display mounted to said handle, said display arranged to display the length of extended tape.

4. The tool according to claim 1, wherein the tape includes any one of: a filament, a stainless steel tape, a wire or a ruler having gradations marked thereon.

5. The tool according to claim 1, wherein the aperture is located on an articulated tip, said articulated tip arranged to selectively direct the tape along an axis different to the axis of the elongate shaft.

6. The tool according to claim 5, further including a pair of cables placed parallel to, and on opposed sides of, the shaft, such that an end of each cable is coupled to the tip adjacent to the aperture and the other end is coupled to a lever positioned on the handle, such that operation of the lever applies a tensile force to one of the cables and consequently pivots the articulated tip.

7. The tool according to claim 1, wherein the extended tape includes a grip at an end of said tape, said grip arranged to engage tissue so as to anchor said end sufficiently anchor the tape and consequently allow withdrawal of the tape on movement of the tool.

8. The tool according to claim 7, wherein said grip comprises any one or combination of: pincher, frictional anchor or a barb.

9. The tool according to claim 1, wherein the tape includes a loop and slip tie, said loop and slip tie arranged to progressively increase the size of the loop as tape is projected from the aperture.

10. The tool according to claim 1, wherein the tape includes a scissor ruler arranged to progressively open as tape is projected from the aperture.

11. The tool according to claim 1, wherein the tape includes a hinged linkage arranged to selectively pivot on application of an offset load to said hinged linkage.

12. The tool according to claim 2, wherein the tape includes a hinged linkage arranged to selectively pivot on application of an offset load to said hinged linkage.

13. The tool according to claim 3, wherein the tape includes a hinged linkage arranged to selectively pivot on application of an offset load to said hinged linkage.

14. The tool according to claim 4, wherein the tape includes a hinged linkage arranged to selectively pivot on application of an offset load to said hinged linkage.

15. The tool according to claim 5, wherein the tape includes a hinged linkage arranged to selectively pivot on application of an offset load to said hinged linkage.

16. The tool according to claim 6, wherein the tape includes a hinged linkage arranged to selectively pivot on application of an offset load to said hinged linkage.

17. The tool according to claim 7, wherein the tape includes a hinged linkage arranged to selectively pivot on application of an offset load to said hinged linkage.

18. The tool according to claim 8, wherein the tape includes a hinged linkage arranged to selectively pivot on application of an offset load to said hinged linkage.
